(19) United States
(12) Patent Application Publication
(10) Pub. No.: US 2023/0218410 A1
(43) Pub. Date: Jul. 13, 2023

(54) Prosthetic Ankle-Foot Device, in Particular of the Biomimetic Type

(71) Applicant: Università Degli Studi Di Padova, Padova (PD) (IT)

(72) Inventors: Nicola Petrone, Noventa Padovana (IT); Paolo Mistretta, Padova (IT); Riccardo Faggian, Dolo (IT)

(21) Appl. No.: 18/007,576

(22) PCT Filed: Jun. 1, 2021
(86) PCT No.: PCT/IB2021/054798

§ 371 Date: Dec. 1, 2022

(30) Foreign Application Priority Data
Jun. 3, 2020 (IT) 102020000013141

(51) Int. Cl. A61F 2/66; A61F 2/76

(52) U.S. Cl. CPC A61F 2/6607; A61F 2/76; A61F 2002/5067

(57) ABSTRACT

A prosthetic ankle-foot device, in particular of the biomimetic type, includes: a tibia element; a talus element movably connected to the tibia element through a first talocrural joint having a first hinge joint; a calcaneus element movably connected to the talus element through a second subtalar joint having a second hinge joint; a medial metatarsal element movably connected to the calcaneus element through a third medial midtarsal joint, the third joint having a third hinge joint; a lateral metatarsal element movably connected to the calcaneus element through a fourth lateral midtarsal joint, the fourth joint having a fourth hinge joint; a medial phalanx element movably connected to the medial metatarsal element through a fifth medial metatarsophalangeal joint, the fifth joint having a fifth hinge joint; a lateral phalanx element movably connected to the lateral metatarsal element through a sixth lateral metatarsophalangeal joint, the sixth joint having a sixth hinge joint; an elastic actuation element having an upper part movably connected to the tibia element and a lower part movably connected to the calcaneus element.

### BACKGROUND OF THE INVENTION

### 1. The Field of the Invention

The present invention relates to a prosthetic ankle-foot device, in particular of the biomimetic type.

### 2. The Relevant Technology

Several types of prosthetic ankle-foot devices are known in the art, which can be divided into three big categories: conventional feet (CF), energy storing and returning feet (ESR), and bionic feet (BIO).

Conventional feet, which were developed first, have no concentrated-type degree of freedom, and cannot therefore make physiological movements; their main function is to damp the forces generated by the impact of the foot on the ground and transfer them to the tibia.

Energy storing and returning feet may be either of the single-joint type, typically having one degree of freedom corresponding to the ankle (talocrural) joint, or of the multi-joint type, wherein the prosthesis can move about different articulation axes, more or less corresponding to physiological movements.

Bionic feet can be divided into two types, i.e., stabilizing (also referred to as “quasi-active”) bionic feet and propulsive (also referred to as “active”) bionic feet. In the former, the active components (usually consisting of motors) perform the function of adjusting some of the prosthesis' features, e.g., ankle rigidity or foot angle, in order to adapt to different sloping grounds. In the latter, the active components perform the function of supplying power to the joints (in particular, the talocrural one), thus causing the prosthesis to move according to a given control logic.

At present, most commercial prosthetic devices fall within the energy storing and returning feet (ESR) category, since only a few stabilizing bionic prostheses and just one propulsive bionic prosthesis is currently available on the market.

Typical commercial prostheses include a main element corresponding to the ankle-foot assembly and made of deformable material, e.g, carbon-fiber sheet, which allows relative movement between the foot and the leg due to the deformability of the material.

Most prostheses currently undergoing research and development have just one degree of freedom, typically associated with the ankle joint (i.e., the talocrural joint), since the foot is usually considered as a secondary, non-functional element.

It is therefore apparent that such prosthetic devices do not permit the achievement of a mechanical behaviour of the prosthesis that is comparable to that of the biologic ankle-foot system, which has been optimized by genetic evolution and therefore represents an optimal trade-off between different functionalities such as, for example, adaptability to different grounds (thanks to the large number of joints), propulsive capability (due to the presence of muscles), and energetic efficiency (due to the presence of elastic elements capable of storing and releasing energy).

It must also be pointed out that people who have suffered amputation and wearers of lower-limb prosthetic devices establish compensation mechanisms which result in gait asymmetry and which may lead to the onset of secondary disorders (osteoarthritis, osteoporosis, and the like) and/or to higher metabolic energy consumption, inevitably resulting in increased fatigue.

In this frame, document U.S. Pat. No. 10,292,840B2 relates to a passive prosthetic device made up of several parts. In fact, the device described in document U.S. Pat. No. 10,292,840B2 comprises a phalanx portion, a metatarsal portion that is movably coupled to the phalanx portion, an ankle portion that is movably connected to the phalanx portion, and a calcaneus portion that is movably coupled to the ankle portion, wherein each coupling between the parts of the device is effected by means of a torsion spring.

Although in document U.S. Pat. No. 10,292,840B2 the foot is considered as a functional element, also the prosthetic device described in such document suffers from a few drawbacks.

In particular, such drawbacks are due to the fact that the phalanx portion and the metatarsal portion made in accordance with the teachings of document U.S. Pat. No. 10,292,840B2 consist of solid, rigid bodies that do not allow the prosthetic device to achieve adequate adaptability to different grounds, especially when such grounds are rough and/or unstable.

Moreover, the coupling of the parts of the device by means of torsion springs does not ensure an adequate mechanical connection between the forefoot joint and the plantar arch, which is a peculiar characteristic of the biologic ankle-foot complex.

### SUMMARY OF THE INVENTION

In this frame, it is the main object of the present invention to provide a prosthetic ankle-foot device, in particular of the biomimetic type, which is so conceived as to overcome the drawbacks of the prior art.

In particular, it is one object of the present invention to provide a prosthetic ankle-foot device so realized as to permit the achievement of a mechanical behaviour which is comparable to that of the biologic ankle-foot system.

It is another object of the present invention to provide a prosthetic ankle-foot device which offers adequate adaptability to different grounds and high energetic efficiency, being realized to provide optimal energy storage and release.

It is a further object of the present invention to provide a prosthetic ankle-foot device so designed as to minimize the establishment of compensation mechanisms by a person wearing said device and to avoid any gait asymmetry, thus preventing the onset of secondary disorders and/or a higher consumption of metabolic energy which would result in increased fatigue.

It is another object of the present invention to provide a prosthetic ankle-foot device so realized as to make it possible to assess the effect of a single factor or parameter on a specific functionality of the prosthetic device.

It is yet another object of the present invention to provide a prosthetic ankle-foot device that is not too expensive to manufacture and difficult to set up.

### BRIEF DESCRIPTION OF THE DRAWINGS

Further objects, features and advantages of the present invention will become apparent in the light of the following detailed description and the annexed drawings, which are supplied by way of non-limiting explanatory example, wherein:

FIG. 1 is a perspective view of a prosthetic ankle-foot device, in particular of the biomimetic type, according to the present invention;

FIG. 2 is an exploded perspective view of the prosthetic device according to the present invention shown in FIG. 1;

FIG. 3 is a lateral view of the prosthetic device according to the present invention;

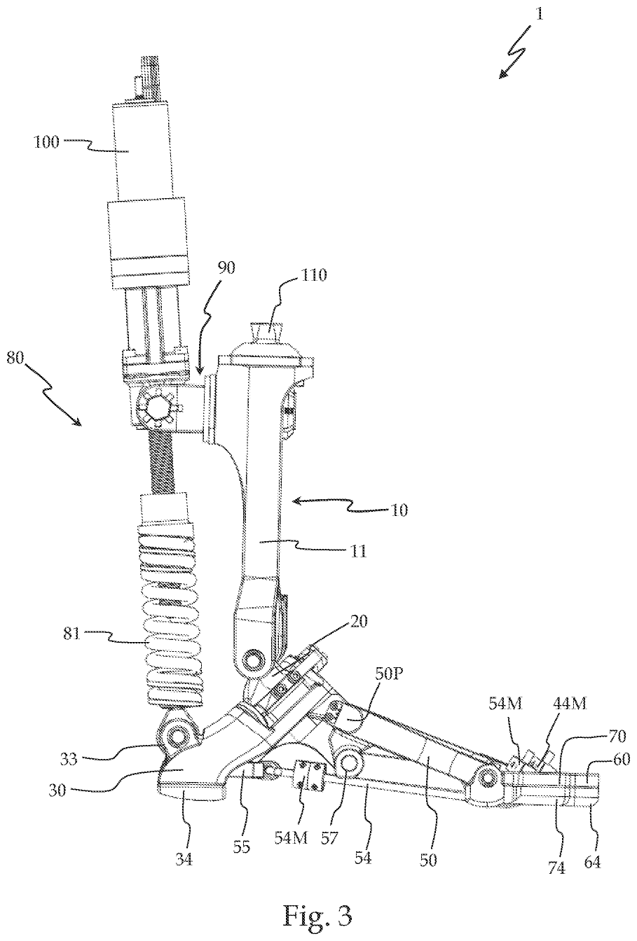

FIG. 4 is an exploded lateral view of the prosthetic device shown in FIG. 3.

### DETAILED DESCRIPTION OF THE PREFERRED EMBODIMENTS

Referring now to the annexed drawings, reference numeral 1 designates as a whole a prosthetic ankle-foot device, in particular of the biomimetic type, according to the present invention.

In accordance with the present invention, the device 1 comprises a tibia element 10, a talus element 20 movably connected to the tibia element 10 through a first talocrural joint A1 comprising a first hinge joint, and a calcaneus element 30 movably connected to the talus element 20 through a second subtalar joint A2 comprising a second hinge joint.

In addition, the device 1 comprises a medial metatarsal element 40 movably connected to the calcaneus element 30 through a third medial midtarsal joint A3, said third joint A3 comprising a third hinge joint, and a lateral metatarsal element 50 movably connected to the calcaneus element 30 through a fourth lateral midtarsal joint A4, said fourth joint A4 comprising a fourth hinge joint.

The device 1 according to the present invention further comprises a medial phalanx element 60 movably connected to the medial metatarsal element 40 through a fifth medial metatarsophalangeal joint A5, said fifth joint A5 comprising a fifth hinge joint, and a lateral phalanx element 70 movably connected to the lateral metatarsal element 50 through a sixth lateral metatarsophalangeal joint A6, said sixth joint A6 comprising a sixth hinge joint.

It should be noted that said hinge joints are preferably implemented as respective hinges, in particular cylindrical hinges.

The device 1 according to the present invention further comprises an elastic actuation element (designated as a whole by reference numeral 80 in the annexed drawings) comprising an upper part movably connected to the tibia element 10 and a lower part movably connected to the calcaneus element 30.

In a preferred embodiment, the tibia element 10 has a fork-like shape and comprises a first arm 11 and a second arm 12 substantially parallel to each other.

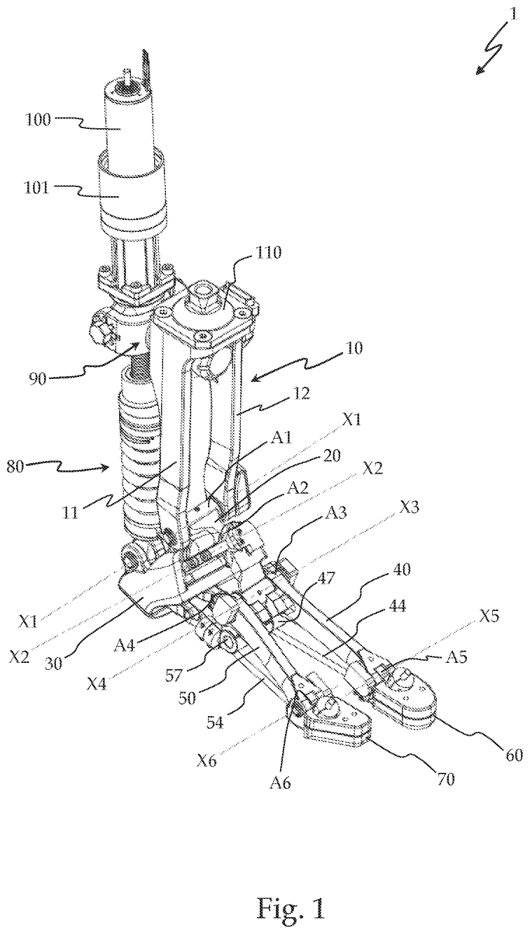

In accordance with the present invention, the first talocrural joint A1 lies on a first axis X1 and the second subtalar joint A2 lies on a second axis X2, wherein said first axis X1 and second axis X2 (shown in FIG. 1) form an oblique ankle dual axis, in particular having biomimetic orientation and position; as a consequence, said oblique dual axis turns out to be similar to the biologic ankle complex, i.e., similar to the physiological talocrural joint and subtalar joint.

In particular, the talocrural axis is inclined relative to the midlateral axis by approximately 10° in the frontal (or coronal) anatomical plane and by approximately 6° in the horizontal (or transversal) plane; as to the subtalar axis, it is inclined relative to the sagittal axis by approximately 42° in the sagittal plane and by approximately 16° in the horizontal plane.

Like its biological counterpart, the first talocrural joint A1 is responsible for the physiological dorsiflexion and plantarflexion movements, i.e., the flexion and extension movements of the ankle; the rigidity of the first talocrural joint A1 is given by the presence of the actuation element 80, and the range of movement of said first talocrural joint A1 is such as to allow different gait types according to a physiological biomechanical pattern, including, without being limited to, walking on flat ground, walking uphill and downhill, and climbing and descending stairs.

In a preferred embodiment, the first talocrural joint A1 comprises a first aperture 11A associated with the first arm 11 of the tibia element 10 and a second aperture 12A associated with the second arm 12 of the tibia element 10, wherein said apertures 11A, 12A are coupled to a talocrural pin 21 integral with the talus element 20; preferably, each one of said apertures 11B, 12B comprises a bushing, in particular of the self-lubricating type.

As far as the second subtalar joint A2 is concerned, it is responsible, just like the corresponding biological joint, for pronation and supination movements.

The second subtalar joint A2 comprises a hole 22 on the talus element 20, which is coupled to a subtalar pin 31 integral with the calcaneus element 30; preferably, said hole 22 houses at least one bushing, in particular of the self-lubricating type.

Moreover, the second subtalar joint A2 is so realized as to have a rigidity of its own. For this purpose, the second subtalar joint A2 comprises a subtalar pad 23 positioned between opposed faces of the talus element 20 and of the calcaneus element 30; preferably, said subtalar pad 23 is made of elastomeric material, and in such a way as to obtain a rigidity of the second subtalar joint A2 that substantially corresponds to that of the biological counterpart.

The particular construction of the subtalar pad 23 makes it possible to specifically design the rigidity curve of the second subtalar joint A2 as needed, even as a non-linear one.

The third medial midtarsal joint A3 and the fourth lateral midtarsal joint A4 are substantially parallel and coaxial to each other, said joints A3, A4 lying on a third axis X3 and a fourth axis X4, respectively, which substantially coincide; for this reason, the third axis X3 and the fourth axis X4 are represented in FIG. 1 as a single dashed-dotted line.

According to a preferred embodiment, the third medial midtarsal joint A3 comprises a first pin 32A integral with the calcaneus element 30, which articulates with a hole 41 that is present on a proximal part of the medial metatarsal element 40, in particular said hole 41 being associated with at least one radial bearing or a self-lubricating bushing.

Furthermore, the fourth lateral midtarsal joint A4 comprises a second pin 32B integral with the calcaneus element 30, which articulates with a hole 51 that is present on a proximal part of the lateral metatarsal element 50, in particular said hole 51 being associated with at least one self-lubricating bushing.

The fifth medial metatarsophalangeal joint A5 and the sixth lateral metatarsophalangeal joint A6 lie on a fifth axis X5 and a sixth axis X6, respectively, which are substantially parallel and not coinciding.

The fifth medial metatarsophalangeal joint A5 consists of a hinge joint comprising a medial metatarsophalangeal pin 42 which articulates with at least one first aperture 43 that is present on a distal part of the medial metatarsal element 40 and with a second aperture 62 that is present on an arm 61 extending from the medial phalanx element 60.

In its turn, the sixth lateral metatarsophalangeal joint A6 consists of a hinge joint comprising a lateral metatarsophalangeal pin 52 which articulates with at least one first aperture 53 that is present on a distal part of the lateral metatarsal element 50 and with a second aperture 72 that is present on an arm 71 extending from the lateral phalanx element 70.

It is therefore clear that the tibia element 10, the talus element 20 and the calcaneus element 30 find a direct biological counterpart in the biological tibia, talus and calcaneus, respectively. As concerns the medial metatarsal element 40, the lateral metatarsal element 50, the medial phalanx element 60 and the lateral phalanx element 70, they do not have a direct biological counterpart, since they are “functional” elements. In fact, the medial metatarsal element 40 mimics the function of the first and second biological metatarsi, while the lateral metatarsal element 50 mimics the third, fourth and fifth biological metatarsi; the same also applies to the medial phalanx element 60 and the lateral phalanx element 70. This division into functional groups allows reducing the complexity of the device 1 according to the present invention, while at the same time maintaining the main functionalities of the corresponding biological elements. In particular, the structure of the device 1 according to the present invention makes it possible to obtain three plantar arches (medial longitudinal, lateral longitudinal and transverse), while at the same time permitting the coupling between the plantar arches (midtarsal joints) and the metatarsophalangeal joints.

In this frame, the first talocrural joint A1 and the second subtalar joint A2 constitute the joints of an ankle complex of the device 1 according to the present invention, and have a direct biological counterpart, in that they mimic its position and spatial orientation.

The remaining four joints (i.e., the third medial midtarsal joint A3, the fourth lateral midtarsal joint A4, the fifth medial metatarsophalangeal joint A5 and the sixth lateral metatarsophalangeal joint A6) constitute the joints of the foot of the device 1 according to the present invention and do not have a direct biological counterpart, in that they are functional joints. In this frame, the two midtarsal joints A3, A4 mimic the function of the biological midtarsal joint (also referred to as Chopart's joint), which is composed of the calcaneocuboid and talonavicular joints, while the two metatarsophalangeal joints A5, A6 mimic the biological function of the five biological metatarsophalangeal joints.

Moreover, said four joints A3, A4, A5, A6 constitute a single functional group, just like in the biological foot, where there is a direct coupling between midtarsal and metatarsophalangeal joints. In particular, the complex formed by the calcaneus element 30, the third medial midtarsal joint A3, the medial metatarsal element 40, the fifth medial metatarsophalangeal joint A5 and the medial phalanx element 60 constitutes the medial longitudinal arch. Likewise, the complex formed by the calcaneus element 30, the fourth lateral midtarsal joint A4, the lateral metatarsal element 50, the sixth lateral metatarsophalangeal joint A6 and the lateral phalanx element 70 constitutes the lateral longitudinal arch. In addition, the transverse (or transversal, or anterior) arch is formed as a consequence of the presence of the medial longitudinal arch and the lateral longitudinal arch between the heads of the medial metatarsal element 40 and of the lateral metatarsal element 50.

As can be seen in the annexed drawings, the actuation element 80 preferably comprises a spring 81, in particular a coil spring, connected to an upper body 82 which articulates with the tibia element 10 through a pivotable fork system allowing two degrees of freedom between said actuation element 80 and the tibia element 10.

In this respect, the pivotable fork system according to the present invention comprises a fork-shaped element 90 and first connection elements which allow a first relative rotational motion between the actuation element 80 and the fork-shaped element 90; note that said first rotational motion constitutes the first degree of freedom between the actuation element 80 and the tibia element 10. In the embodiment shown in the annexed drawings, said first connection elements comprise radial bearings 82C associated with the upper body 82 of the actuation element 80, which articulates with respective pins 91P associated with the arms 91 of the fork-shaped element 90.

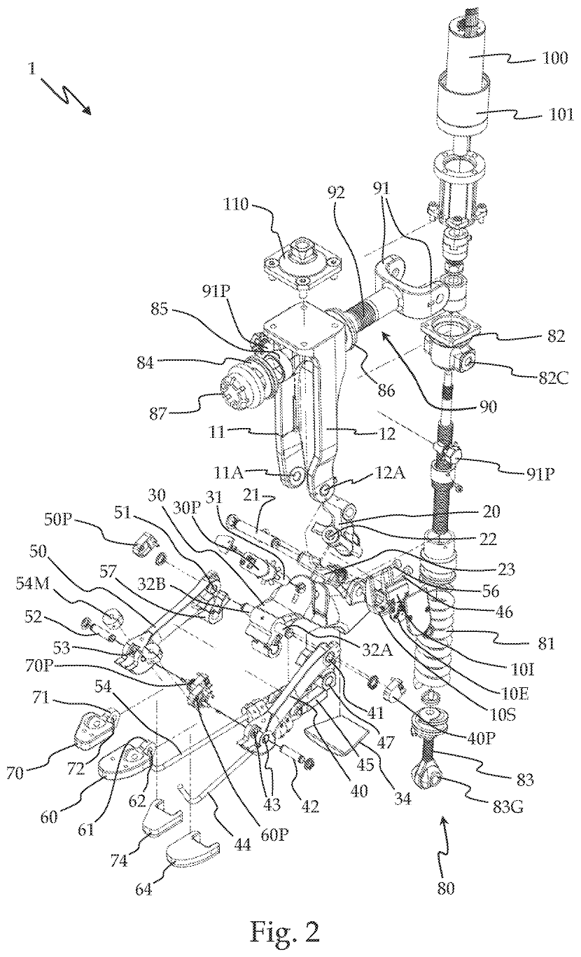

Furthermore, the pivotable fork system according to the present invention comprises second connection elements (designated by reference numerals 84, 85, 86 and 87 in FIG. 2), which allow a second relative rotational motion between the actuation element 80 and the fork-shaped element 90; note that said second rotational motion constitutes the second degree of freedom between the actuation element 80 and the tibia element 10. Preferably, said second connection elements comprise at least one bearing 84, 85, 86 mounted to a rod 92 of the fork-shaped element 90, which articulates with at least one respective seat (not shown in the annexed drawings) associated with the tibia element 10.

In the embodiment shown in FIG. 2, one can see that said second connection elements comprise a plurality of bearings, i.e.:
a first axial needle bearing 84 acting axially between a circular crown that is present in the tibia element 10 and a corresponding area that is present on the castellated nut 87;
a radial needle bearing 85 acting radially in a hole of the tibia element 10 in which the rod 92 of the fork-shaped element 90 is housed;
a second axial needle bearing 86 acting axially between the circular crown that is present in the tibia element 10 and a front face of the fork-shaped element 90.

Said second connection elements comprise also a locking element 87 which is coupled to the rod 92 of the fork-shaped element 90 to tighten the bearings 84, 85, 86 against the tibia element 10; when observing FIG. 2, it can be noticed that said locking element 87 may consist of a castellated nut, possibly associated with a cotter pin (not shown) to prevent it from turning about said rod 92.

The lower part of the actuation element 80 articulates with the calcaneus element 30 through a terminal 83 that allows two degrees of freedom between said actuation element 80 and calcaneus element 30. In the embodiment shown in the annexed drawings, said terminal 83 articulates with a fork 33 of the calcaneus element 30 and comprises a ball joint 83G associated with anti-torsion elements; in particular, said anti-torsion elements comprise lateral faces of the ball joint 83G provided with elements tangent to the inner faces of said fork 33. Such an embodiment permits locking exclusively an undesired third degree of freedom of the ball joint 83G, i.e., the degree of freedom corresponding to torsion of the actuation element 80.

In accordance with a preferred embodiment, the device 1 comprises a motor 100, in particular of the electric type, associated with the upper body 82 of said actuation element 80, possibly through the interposition of a reducer 101.

It is clear that, due to the provision of the motor 100, the prosthetic device 1 of the present invention becomes active, since it can produce net positive power.

In this context, the assembly made up of the actuation element 80 and the motor 100 reproduces the positioning (i.e., the origin and the insertion) of the biological soleus muscle, which is a biarticular muscle because it acts simultaneously upon both the talocrural joint and the subtalar joint. It should be noted that the particular implementation shown in the annexed drawings permits minimizing the peak power required from the motor 100 in order to cause the device 1 to make the walking gesture.

Furthermore, the assembly made up of the actuation element 80 and the motor 100 reproduces the functionalities of the whole set of biological plantarflexor and dorsiflexor muscles; however, unlike biological muscles, this assembly can operate in a desmodromic manner both when pulling (plantarflexion) and when pushing (dorsiflexion), and can produce all the power of the complex of plantarflexor (mainly soleus, medial and lateral gastrocnemius) and dorsiflexor (mainly anterior tibial) muscles.

It should be noted that, in accordance with the teachings of the present invention, the actuation element 80 is positioned in series with the motor 100, just like the tendon is in series with the active element (muscle) in the biological counterpart.

In accordance with a preferred embodiment, the rigidity of the actuation element 80, in particular the spring 81, must be such as to provide a trade-off among: minimization of the peak power of the electric motor during the walking cycle, minimization of the energy consumed by the electric motor during the walking cycle, and possibility of using the prosthesis with a locked motor (motor turned off). It has been observed, in fact, that the optimal rigidity of the active actuation system including the motor 100 (minimization of peak power and energy consumption) corresponds, with due approximation, to the optimal rigidity that would be necessary if the device 1 did not include the motor 100 and were, as a consequence, totally passive (linear regression of the torque vs. ankle angle curve). Note that such optimal rigidity also corresponds, with due approximation, to the physiological rigidity of the Achilles tendon (tendon of the plantarflexor muscles).

The prosthetic device 1 according to the present invention can therefore be made to operate correctly also in “passive mode”, by preventing the rotation of the electric motor 100 or by building the device 1 without associating the motor 100 with the actuation element 80.

From the above description it clearly emerges that the kinematic mechanism formed by the tibia element 10, the talus element 20, the calcaneus element 30, the actuation element 80 and the fork-shaped element 90, possibly also including the motor 100, constitutes a closed-chain spatial kinematic mechanism, as opposed to a planar kinematic mechanism like those of prior-art devices.

In this context, the pivotable fork system with two degrees of freedom that connects the upper part of the actuation element 80 to the tibia element 10 and the ball joint reduced to a double cylindrical joint that connects the lower part of the actuation element 80 to the calcaneus element 30 perform a dual function. Firstly, such an embodiment ensures a smooth actuation of a system with two degrees of freedom, made up of the first talocrural joint A1 and the second subtalar joint A2 coupled together; it is thus possible to actuate said system with two degrees of freedom between the tibia element 10 and the calcaneus element 30 (as in the biological ankle-foot complex), instead of actuating one degree of freedom at a time with planar mechanisms. Secondly, such an embodiment makes it possible (even though if the mechanism is a spatial kinematic mechanism) to obtain the previously described desmodromic system, i.e., a system capable of both pulling and pushing.

In accordance with the present invention, the device 1 comprises:
a first elastic element 44, which connects the calcaneus element 30 to the medial phalanx element 60 and which is positioned under the medial metatarsal element 40,
a second elastic element 54, which connects the calcaneus element 30 to the lateral phalanx element 70 and which is positioned under the lateral metatarsal element 50.

The connection between the elastic elements 44, 54 and the calcaneus element 30, the medial phalanx element 60 and the lateral phalanx element 70 is accomplished through suitable fastening means, e.g., respective clamps 44M, 54M.

Said elastic elements 44, 54 perform the task of reproducing the function of the biological plantar aponeurosis, in that they confer rigidity on the (medial and lateral) longitudinal plantar arches and permit the coupling between the midtarsal joints A3, A4 and the metatarsophalangeal joints A5, A6.

In a preferred embodiment, the device 1 comprises a first system 45, 46 for adjusting the tension of the first elastic element 44 and a second system 55, 56 for adjusting the tension of the second elastic element 54.

In particular, the first adjustment system comprises a first tensioner 45 connected to the first elastic element 44 and associated with a first adjustment screw 46 for causing the first tensioner 45 to slide relative to the calcaneus element 30 in order to either increase or decrease the tension of the first elastic element 44.

Furthermore, the second adjustment system comprises a second tensioner 55 connected to the second elastic element 54 and associated with a second adjustment screw 56 for causing the second tensioner 55 to slide relative to the calcaneus element 30 in order to either increase or decrease the tension of the second elastic element 54.

Said adjustment systems make it possible to calibrate and adjust the rigidity curve of the plantar arches and of the metatarsophalangeal joints A5, A6 on the basis of several factors, such as, for example, the person's weight, the mode of operation of the device 1 (walking on flat ground, walking on rough ground, etc.), or according to the user's preferences.

Preferably, the underside of the medial metatarsal element 40 comprises a first arm 47, whereon the first elastic element 44 rests, and the underside of the lateral metatarsal element 50 comprises a second arm 57, whereon the second elastic element 54 rests.

Said first arm 47 and second arm 57 have several purposes, in that they allow:
preventing the plantar arch from collapsing, because, as the arch lowers, they will keep substantially constant the leverage ratio between the elastic elements 44, 54 and the midtarsal joints A3, A4;
accurately setting the rigidity of the plantar arch (and hence of the metatarsophalangeal joints A5, A6) by appropriately designing the kinematic mechanism;
leaving sufficient room for inserting elements suitable for locking the midtarsal joints A3, A4, as will be described hereinafter.

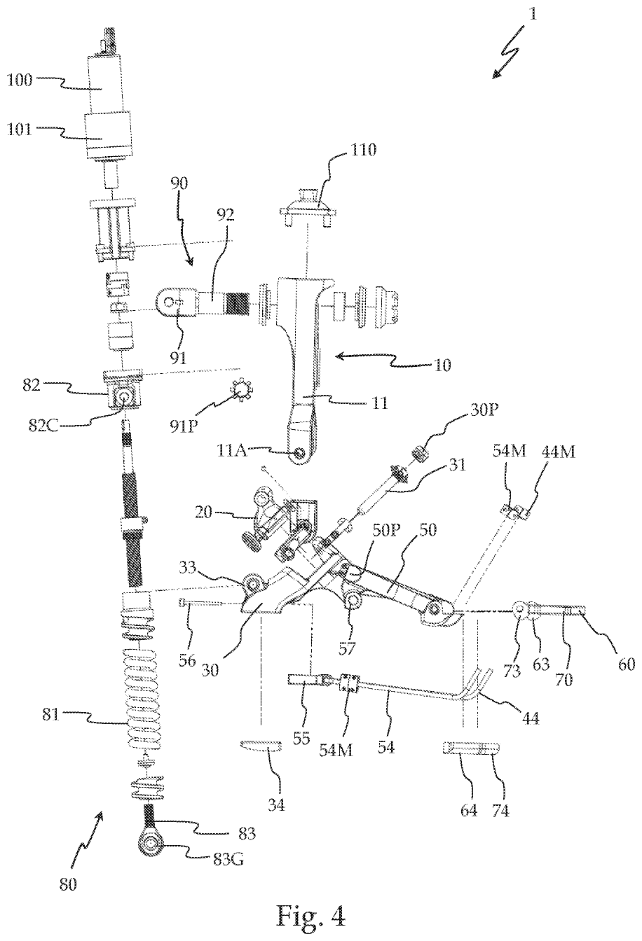

In accordance with a preferred embodiment, the medial phalanx element 60 comprises a first cam 63 (visible in FIG. 4), whereon the first elastic element 44 rests, and the lateral phalanx element 70 comprises a second cam 73 (also visible in FIG. 4), whereon the second elastic element 54 rests, wherein said first cam 63 and second cam 73 may be so realized as to accurately design the rigidity curve of the fifth medial metatarsophalangeal joint A5 and of the sixth lateral metatarsophalangeal joint A6. In substance, by changing the radius of the cams 63, 73 it is possible to modify the torsional rigidity of the respective joints A5, A6.

Preferably, the device 1 according to the present invention is so realized as to comprise at least one locking element for each one of the foot joints A3, A4, A5, A6.

In particular, the device 1 may comprise:
a first pin (not shown in the drawings) passing through a hole in the first arm 47 and through another hole in the calcaneus element 30 for locking the third medial midtarsal joint A3;
a second pin (not shown in the drawings) passing through a hole in the second arm 57 and through another hole in the calcaneus element 30 for locking the fourth lateral midtarsal joint A4;
a first strap, in particular nail-shaped (not shown in the drawings), fixed between the distal part of the medial metatarsal element 40 and the medial phalanx element 60 for locking the fifth medial metatarsophalangeal joint A5;
a second strap, in particular nail-shaped (not shown in the drawings), fixed between the distal part of the lateral metatarsal element 50 and the lateral phalanx element 70 for locking the sixth lateral metatarsophalangeal joint A6.

The possibility of selectively locking the foot joints A3, A4, A5, A6 allows for accurate and detailed quantification of the influence that each one of them has on the functions of interest of the device 1.

In accordance with a preferred embodiment, the device 1 according to the present invention is realized to comprise at least one sensor 10E, 10I, 30P, 40P, 50P, 60P, 70P associated with at least one joint A1, A2, A3, A4, A5, A6 for providing a direct reading of the data concerning such joint.

In particular, the device 1 according to the present invention is preferably so realized as to comprise one or more of the following sensors (especially visible in FIG. 2):
an encoder 10E, in particular of the magnetic type, fastened to the tibia element 10 at the first joint A1 for reading the talocrural joint angle, in particular said encoder 10E being positioned inside a removable support 10S fastened to the tibia element 10 by fastening means;
an inertial sensor 10I fastened to the tibia element 10 at the first talocrural joint A1 for reading the absolute tibia angles, in particular said inertial sensor 10I being housed in the same support 10S as the encoder 10E;
a first potentiometer 30P fastened to the calcaneus element 30 at the second joint A2 for reading the subtalar joint angle;
a second potentiometer 40P fastened to the medial metatarsal element 40 at the third joint A3 for reading the medial midtarsal angle;
a third potentiometer 50P fastened to the lateral metatarsal element 50 at the fourth joint A4 for reading the lateral midtarsal angle;
a fourth potentiometer 60P fastened at the fifth joint A5 for reading the medial metatarsophalangeal angle, in particular said fourth potentiometer 60P being fastened to the medial metatarsal element 40;
a fifth potentiometer 70P fastened at the sixth joint A6 for reading the lateral metatarsophalangeal angle, in particular said fifth potentiometer 70P being fastened to the lateral metatarsal element 50.

The measurements taken by the sensors 10E, 10I, 30P, 40P, 50P, 60P, 70P permit obtaining a direct reading in real time of all six (relative) joint angles, as well as reading the absolute angles with respect to the global reference system of the tibia element 10, e.g., in terms of pitch and roll.

The data thus obtained can be used for different purposes, including the creation of robust (because based on multiple input data) control models for the motor 100, the creation of regression models for estimating all the kinematic and kinetic quantities of the prosthetic device 1 (such as ground reaction forces, center of pressure trajectory, etc.), the evaluation of the quality of the gait of the person using the prosthetic device 1, and so forth.

In accordance with a preferred embodiment, the device 1 according to the present invention comprises at least one interface element 34, 64, 74, in particular made of rubber or a similar material, for damping the ground impact forces and ensuring optimal grip on the ground.

In particular, said at least one interface element comprises one or more of the following elements:
a first interface element 34 fixed under the calcaneus element 30;
a second interface element 64 fixed under the medial phalanx element 60;
a third interface element 74 fixed under the lateral phalanx element 70.

It is therefore clear that said interface elements 34, 64, 74 are similar to the fat pads found in the biological foot.

The device 1 according to the present invention has a volumetric size and a mass which are similar to those of the biological counterpart, so that it can be used by amputated people even inside normal footwear; moreover, the device 1 is easily scalable for obtaining different sizes comparable to those of the biological foot.

The device 1 may be so realized as to comprise a covering, in particular made of silicone-based material, into which said device 1 can be fitted.

The device 1 according to the present invention further comprises a coupling element 110, in particular of the pyramidal type, which permits connecting the device 1 to a tibial pylon (not shown in the annexed drawings); preferably, said coupling element 110 is fixed to the top part of the tibia element 10.

The features of the prosthetic ankle-foot device 1, in particular of the biomimetic type, according to the present invention, as well as the advantages thereof, are apparent from the above description.

In fact, the device 1 according to the present invention is so realized as to permit the achievement of a mechanical behaviour which is comparable to that of the biological ankle-foot system.

Furthermore, due to the provisions of the present invention, the prosthetic ankle-foot device 1 offers good adaptability to different grounds and high energetic efficiency, being designed to provide propulsive capability as well as adequate energy storage and release.

The peculiar features of the device 1 according to the present invention make it possible to minimize the establishment of compensation mechanisms by a wearer of said device; in particular, the prosthetic ankle-foot device 1 prevents gait asymmetry, thus avoiding the onset of secondary disorders and/or a higher consumption of metabolic energy that would result in increased fatigue.

The provision of the prosthetic ankle-foot device 1 with sensors 10E, 10I, 30P, 40P, 50P, 60P, 70P makes it possible to accurately quantify the effects of single factors or parameters on a specific function of interest of the device 1.

The prosthetic ankle-foot device 1 according to the present invention is not very expensive and is easy to set up, in addition to having a volumetric size and a mass that are similar to those of its biological counterpart, which allow it to be used by amputated people also inside normal footwear; moreover, the device 1 is easily scalable for obtaining different sizes comparable to those of the biological foot.

The prosthetic ankle-foot device 1, in particular of the biomimetic type, described herein by way of example may be subject to many possible variations without departing from the novelty spirit of the inventive idea; it is also clear that in the practical implementation of the invention the illustrated details may have different shapes or be replaced with other technically equivalent elements.

It can therefore be easily understood that the present invention is not limited to the above-described prosthetic ankle-foot device 1, but may be subject to many modifications, improvements or replacements of equivalent parts and elements without departing from the inventive idea, as clearly specified in the following claims.

1. A prosthetic ankle-foot device, in particular of the biomimetic type, said device comprising:
a tibia element;
a talus element movably connected to the tibia element through a first talocrural joint comprising a first hinge joint;
a calcaneus element movably connected to the talus element through a second subtalar joint comprising a second hinge joint;
a medial metatarsal element movably connected to the calcaneus element through a third medial midtarsal joint, said third joint comprising a third hinge joint;
a lateral metatarsal element movably connected to the calcaneus element through a fourth lateral midtarsal joint said fourth joint comprising a fourth hinge joint;
a medial phalanx element movably connected to the medial metatarsal element through a fifth medial metatarsophalangeal joint, said fifth joint comprising a fifth hinge joint;
a lateral phalanx element movably connected to the lateral metatarsal element through a sixth lateral metatarsophalangeal joint, said sixth joint comprising a sixth hinge joint;
an elastic actuation element comprising an upper part movably connected to the tibia element and a lower part movably connected to the calcaneus element.

2. The device according to claim 1, wherein the tibia element has a fork-like shape and comprises a first arm and a second arm substantially parallel to each other.

3. The device according to claim 1, wherein the first talocrural joint lies on a first axis and the second subtalar joint lies on a second axis, wherein said first axis and second axis form an oblique ankle dual axis having biomimetic orientation and position.

4. The device according to claim 3, wherein the first talocrural joint comprises a first aperture associated with the first arm of the tibia element and a second aperture associated with the second arm of the tibia element, wherein said apertures are coupled to a talocrural pin integral with the talus element.

5. The device according to claim 1, wherein the second subtalar joint comprises a hole on the talus element, which is coupled to a subtalar pin integral with the calcaneus element.

6. The device according to claim 1, wherein the second subtalar joint comprises a subtalar pad positioned between opposed faces of the talus element and of the calcaneus element, said subtalar pad being made of elastomeric material.

7. The device according to claim 1, wherein the third medial midtarsal joint and the fourth lateral midtarsal joint are substantially parallel and coaxial to each other, said joints lying on a third axis and a fourth axis, respectively, which substantially coincide.

8. The device according to claim 7, wherein the third medial midtarsal joint comprises a first pin integral with the calcaneus element, which articulates with a hole that is present on a proximal part of the medial metatarsal element.

9. The device according to claim 7, wherein the fourth lateral midtarsal joint comprises a second pin integral with the calcaneus element, which articulates with a hole that is present on a proximal part of the lateral metatarsal element.

10. The device according to claim 1, wherein the fifth medial metatarsophalangeal joint and the sixth lateral metatarsophalangeal joint lie on a fifth axis and a sixth axis, respectively, which are substantially parallel and not coinciding.

11. The device according to claim 10, wherein the fifth medial metatarsophalangeal joint consists of a hinge joint comprising a medial metatarsophalangeal pin which articulates with at least one first aperture that is present on a distal part of the medial metatarsal element and with a second aperture that is present on an arm extending from the medial phalanx element.

12. The device according to claim 10, wherein the sixth lateral metatarsophalangeal joint consists of a hinge joint comprising a lateral metatarsophalangeal pin which articulates with at least one first aperture that is present on a distal part of the lateral metatarsal element and with a second aperture that is present on an arm extending from the lateral phalanx element.

13. The device according to claim 1, wherein the actuation element comprises a spring connected to an upper body which articulates with the tibia element through a pivotable fork system allowing two degrees of freedom between said actuation element and the tibia element.

14. The device according to claim 13, wherein said pivotable fork system comprises:
a fork-shaped element,
first connection elements which allow a first relative rotational motion between the actuation element and the fork-shaped element,
second connection elements which allow a second relative rotational motion between the actuation element and the fork-shaped element.

15. The device according to claim 14, wherein said first connection elements comprise radial bearings associated with the upper body of the actuation element, which articulate with respective pins associated with the arms of the fork-shaped element.

16. The device according to claim 14, wherein said second connection elements comprise at least one bearing mounted to a rod of the fork-shaped element, which articulates with at least one respective seat associated with the tibia element.

17. The device according to claim 1, wherein the lower part of the actuation element articulates with the calcaneus element through a terminal that allows two degrees of freedom between said actuation element and calcaneus element.

18. The device according to claim 17, wherein said terminal articulates with a fork of the calcaneus element and comprises a ball joint associated with anti-torsion elements, said anti-torsion elements comprising lateral faces of the ball joint provided with elements tangent to the inner faces of said fork.

19. The device according to claim 1, wherein said device comprises a motor associated with the upper body of said actuation element through the interposition of a reducer.

20. The device according to claim 1, wherein said device comprises:
a first elastic element, which connects the calcaneus element to the medial phalanx element and which is positioned under the medial metatarsal element,
a second elastic element, which connects the calcaneus element to the lateral phalanx element and which is positioned under the lateral metatarsal element.

21. The device according to claim 1, wherein said device comprises a first system for adjusting the tension of the first elastic element and a second system for adjusting the tension of the second elastic element.

22. The device according to claim 1, wherein the underside of the medial metatarsal element comprises a first arm, whereon the first elastic element rests, and the underside of the lateral metatarsal element comprises a second arm, whereon the second elastic element rests.

23. The device according to claim 20, wherein the medial phalanx element comprises a first cam, whereon the first elastic element rests, and the lateral phalanx element comprises a second cam, whereon the second elastic element rests.

24. The device according to claim 1, wherein said device comprises a locking element for the third joint and/or for the fourth joint and/or for the fifth joint and/or for the sixth joint.

25. The device according to claim 1, wherein said device comprises at least one sensor associated with at least one joint for providing a direct reading of the data concerning said joint.

26. The device according to claim 25, wherein said device comprises one or more of the following sensors:
an encoder, of the magnetic type, fastened to the tibia element at the first joint for reading the talocrural joint angle, said encoder being positioned inside a support fastened to the tibia element by fastening means;
an inertial sensor fastened to the tibia element at the first talocrural joint for reading the absolute tibia angles, said inertial sensor being housed in the support of the encoder;
a first potentiometer fastened to the calcaneus element at the second joint for reading the subtalar joint angle;
a second potentiometer fastened to the medial metatarsal element at the third joint for reading the medial midtarsal angle;
a third potentiometer fastened to the lateral metatarsal element at the fourth joint for reading the lateral midtarsal angle;
a fourth potentiometer fastened at the fifth joint for reading the medial metatarsophalangeal angle, said fourth potentiometer being fastened to the medial metatarsal element;
a fifth potentiometer fastened at the sixth joint for reading the lateral metatarsophalangeal angle, said fifth potentiometer being fastened to the lateral metatarsal element.

27. The device according to claim 1, wherein said device comprises at least one interface element, particular made of rubber, for damping the ground impact forces and ensuring optimal grip on the ground.

28. The device according to claim 1, wherein said device comprises a coupling element, of the pyramidal type, which makes it possible to connect the device to a tibial pylon.
